(19) United States
(12) Patent Application Publication
(10) Pub. No.: US 2015/0038671 A1
(43) Pub. Date: Feb. 5, 2015

(54) Efficient Synthesis of CN2097 and RC7 and Their Analogs

(71) Applicants: Keykavous Parang, Irvine, CA (US); Rakesh Tiwari, Irvine, CA (US)

(72) Inventors: Keykavous Parang, Irvine, CA (US); Rakesh Tiwari, Irvine, CA (US)

(21) Appl. No.: 14/292,745

(22) Filed: May 30, 2014

Related U.S. Application Data
(60) Provisional application No. 61/828,941, filed on May 30, 2013.

(51) Int. Cl. C07K 7/56

(52) U.S. Cl. CPC C07K 7/56
USPC 530/317

(57) ABSTRACT

Synthesized macrocyclic ligand, CN2097 and analogs, optimized with systemic structure modifications to develop the compounds with lower molecular weights and less peptidic characters.

### PRIORITY

The present application claims the benefit of U.S. Provisional Patent Application No. 61/828,941 filed on May 30, 2013, the contents of which is hereby incorporated by reference in its entirety.

### BACKGROUND OF THE INVENTION

The present invention generally relates to providing a synthesized macrocyclic ligand that may be optimized with systemic structure modifications to develop the compounds with lower molecular weights and less peptidic characters.

One such example of conjugation is pegylation that not only improves the delivery of the therapeutic molecule, it also changes the pharmacokinetics and pharmacodynamics of the molecules. Pegylation may decrease cellular peptide clearance by reducing elimination through the reticuloendothelial system by specific cell-protein interaction. Pegylation is carried out by using various PEG molecules of different lengths (4-8) to afford stable derivatives of the lead macrocycles.

Yet another example of conjugation is N-terminal lipidation that has been applied to various peptides using myristoyl (C14 carbon chain) as fatty chain to enhance cellular permeability. Conjugation with various lipophilic fatty acyl chains (C12-C20) (lipidation) is an attractive method to improve the cell permeability. Another element is that the lipophilic chain is hydrolyzed intracellularly by hydrolytic enzymes thereby releasing the active parent analog.

Alternatively, a number of cell-penetrating peptides have been used for delivery application of various drugs with great success. HIV-1 Tat protein (Tat:49-57) has shown a great promise by transporting various molecules inside cells. For example, PDZ domain inhibitors can be conjugated with positively-charged poly arginine residues. The oligomer of arginine with 7-9 residues is also an effective transporter. The linking of a polyarginine peptide through a hydrolyzable linker (disulfide bond) to the macrocycle of PDZ domain inhibitors led to the synthesis of novel CN 2097 (as shown in FIG. 1) that generated biological activities intracellularly in the neuronal cells. The mechanism of uptake by these polyarginine based-peptides is by endosomal pathway. The cell penetrating peptide has basic or cationic amino acid, which is responsible for the interaction with cell membrane.

The szeto-schillar peptide (H-Dimethyl tyrosine-[D]-Arg-Phe-Lys-NH₂) has been found to target mitochondria inside the cells. Linking of the peptide to PDZ inhibitor macrocycle using a similar disulfide-disulfide linkage will also be investigated. The szeto-CN2097 conjugate will have lower molecular weight and different biological profile versus conventional cell-penetrating peptide derivative e.g. CN2097.

### BRIEF DESCRIPTION OF THE DRAWINGS

The following detailed description may be further understood with reference to the accompanying drawings in which:

FIG. 1 shows an illustrative diagrammatic view of the linking of a polyarginine peptide through a hydrolyzable linker (disulfide-disulfide bond) to the macrocycle of PDZ domain inhibitors in accordance with the prior art;

FIG. 2 shows an illustrative diagrammatic view of the synthesis of cyclic peptides in accordance with an embodiment of the present invention;

FIG. 3 shows an illustrative diagrammatic view of a detailed synthetic methodology for the synthesis of a disulfide-disulfide analogue of macrocyclic peptide in accordance with an embodiment of the present invention;

FIG. 4 shows an illustrative diagrammatic view of structures of examples of synthesized peptides in accordance with an embodiment of the present invention;

FIG. 5 shows an illustrative diagrammatic view of structures of examples of synthesized peptides with reduced peptidic nature of macrocycle in accordance with an embodiment of the present invention;

FIG. 6 shows an illustrative diagrammatic view of an example of the modification of the peptidic bond to thioamide in accordance with an embodiment of the present invention;

FIG. 7 shows an illustrative diagrammatic view of examples of synthesized peptides with various functional group substitutions in accordance with an embodiment of the present invention;

FIG. 8 shows an illustrative diagrammatic view of an example of a lipidation macrocyclic PDZ domain inhibitor synthesized in accordance with an embodiment of the present invention;

FIG. 9 shows an illustrative diagrammatic view of an example of a szeto-Schillar peptide that was synthesized by Fmoc-t/Bu solid phase in accordance with an embodiment of the present invention;

FIG. 10 shows an illustrative diagrammatic view of an example of Szeto-CN2097 an example of the synthesis of a lead macrocyclic CN 2097 in accordance with an embodiment of the present invention;

FIG. 11 shows an illustrative diagrammatic view of an example of the synthesis of a lead macrocyclic CN 2097 in accordance with an embodiment of the present invention;

FIG. 12 shows an illustrative diagrammatic view of examples of oligocarbamates as alternative carriers in accordance with an embodiment of the present invention;

FIG. 13 shows an illustrative graphical view of concentrations versus average area under the curve for assessing a detection limit of CN2097 in accordance with an embodiment of the present invention;

FIGS. 14-17 shows illustrative diagrammatic views of examples of polyargine disulfide peptides synthesized in accordance with an embodiment of the present invention;

FIG. 18 shows an illustrative diagrammatic view of an example of a series of peptide sequence RCRnC where n=2-6 synthesized in accordance with an embodiment of the present invention;

FIG. 19 shows an illustrative diagrammatic view of an example of the use of a standard Fmoc-based protocol used to synthesize peptide in accordance with an embodiment of the present invention;

FIG. 20 shows an illustrative diagrammatic view of examples of proposed structures that provide new conformations in the sequence of the lead peptide in accordance with an embodiment of the present invention;

FIG. 21 shows an illustrative diagrammatic view of an example of a modification of peptidic bond to ketone, thioamide or reverse amide in accordance with embodiment of the present invention;

FIG. 22 shows an illustrative diagrammatic view of an example of a peptoid synthesized in accordance with an embodiment of the present invention;

FIG. 23 shows an illustrative diagrammatic view of an example of an oligocarbamate synthesized in accordance with an embodiment of the present invention;

FIG. 24 shows an illustrative diagrammatic view of an example of a myristoyl derivative of lead peptide synthesized in accordance with an embodiment of the present invention;

FIG. 25 shows an illustrative diagrammatic view of an example of a Szeto peptide sequence synthesized in accordance with an embodiment of the present invention; and

FIG. 26 shows an illustrative diagrammatic view of a pegylated derivative of R₇Cs-sC synthesized in accordance with an embodiment of the present invention.

### DETAILED DESCRIPTION

In the present invention, synthesized macrocyclic ligand can be optimized with systemic structure modifications to develop the compounds with lower molecular weights and less peptidic characters. The macrocyclic PDZ domain inhibitor can be optimized by truncation from N-terminal to C-terminal, by use of unnatural amino acid (D amino acid), and by changing the peptide backbone to ketone (COCH₃), thioamide (CS—NH) or reverse amide (NH—CO). These proposed structural modifications will provide new insights into development of the potent PDZ domain inhibitors. After initial lead compounds were discovered, they are conjugated with cell-penetrating functional groups for enhancing their cellular uptake.

### Example 1

### Synthesis of Macrocycle CN2097

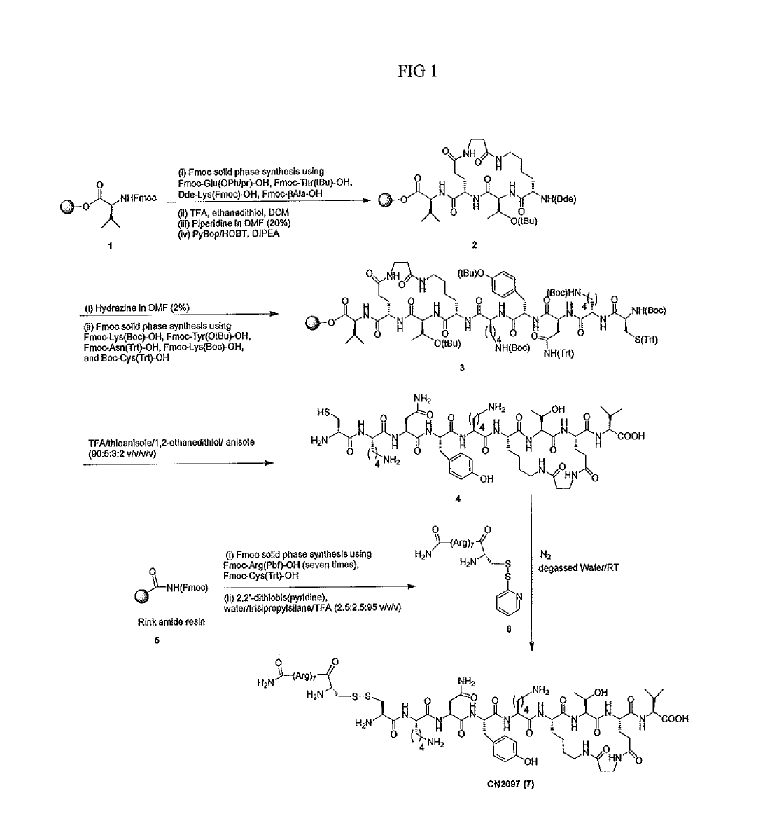

A standard Fmoc-based protocol was used to synthesize a macrocycle targeting the PDZ domain of PSD-95 as previously reported (FIG. 1). The peptide, K₁N₂Y₃K₄K₅T₆E₇V₈, based on the C-terminal residues of CRIPT, was synthesized using Fmoc/tBu solid-phase chemistry. Fmoc-Val-Wang resin was used as a solid-phase resin. The peptide chain was assembled on the Fmoc-Val-Wang resin (1) using coupling and deprotection cycles with HBTU/DIPEA and piperidine in DMF (20%), respectively. After synthesizing the linear protected peptide on the solid phase (Dde-K-(Fmoc)-T(tBu)-E(OPhipr)-V-Wang resin), the Fmoc group, which was at the side chain of the lysine (K₅) was selectively deprotected and was further coupled with Fmoc-β-alanine, which was subsequently cyclized after deprotection of glutamic acid (E₇) side chain to afford 2. The Dde group of N-terminal of peptide 2 on solid phase was deprotected using 2% hydrazine in DMF and was coupled with Fmoc-Lys(Boc)-OH, Fmoc-Tyr(OtBu)-OH, Fmoc-Asn(Trt)-OH, Fmoc-Lys(Boc)-OH, and Boc-Cys(Trt)-OH, respectively to generate 3. The deprotection of the side chains, final cleavage from the solid support using cleavage cocktail R (TFA/thioanisole/1,2-ethanedithiol/anisole 90:5:3:2 v/v/v/v, 2 h), purification with HPLC, and lyophilization afforded cyclic peptide 4 containing a cysteine residue. The chemical structure of peptide 4 was confirmed using a high-resolution time-of-flight electrospray mass spectrometer.

In a separate synthesis, polyarginine-cysteine peptide 6 was synthesized using Fmoc/tBu solid phase chemistry and rink amide resin. By using appropriate cycles of coupling and deprotection, the linear peptide (NH₂—C(Trt)-R (Pbf)₇-rink amide resin) was assembled on solid phase with the cysteine at the N-terminal required for disulfide-disulfide coupling. The final deprotection and cleavage of the peptide from the resin using the 2,2′-dithiobis(pyridine) in cleavage cocktail (water/triisopropylsilane/TFA (2.5:2.5:95, v/v/v) afforded the activated cysteine generated in situ within the polyarginine peptide 6 after HPLC purification and lyophilization.

The final disulfide-disulfide coupling was carried out by conjugation of the cyclic peptide (4, 1 equiv) and the polyarginine peptide (6, 1 equiv) under nitrogen degassed water for 18 h. After final HPLC purification and lyophilization CN2097 (7) containing the disulfide bond was obtained (FIG. 1). The chemical structure of CN2097 was confirmed using a high-resolution time-of-flight electrospray mass spectrometer.

The commercial advantages of the new more efficient synthesis compared to previous work are as follows. First, the final conjugation of cyclic peptide with poly-Arg peptide was carried out in solution phase. Therefore, only an equimolar amount of cyclic peptide is needed as compared to poly-Arg peptide. Solid-phase synthesis requires multiple equivalents of cyclic peptide. Second, higher yield was obtained as compared to reported solid phase method. Third, the final reaction is a single step and therefore does not need multiple protection steps. Four, this method can be easily and successful scaled for the synthesis from 10 mg to 500 mg. Finally, the gram quantities of CN2097 can be made using flash chromatography system to help with initial purification of cyclic and linear polyarginine peptide.

### Example 2

### General Methods for Peptide Synthesis

All the reagents for peptide synthesis, including Fmoc-amino acids, Fmoc-Val-Wang resin, coupling reagents, and Fmoc-amino acid building blocks were purchased from Novabiochem. Other chemicals and reagents were purchased from Sigma Aldrich Chemical Co. (Milwaukee, Wis.). All reactions were carried out in Bio-Rad polypropylene columns by shaking and mixing using a Glass Col small tube rotator in dry conditions at room temperature unless otherwise stated. In general, all peptides were synthesized by the solid-phase synthesis strategy employing Fmoc-based chemistry and Fmoc-L-amino acid building blocks. HBTU and DIPEA in DMF were used as coupling and activating reagents, respectively. Fmoc-deprotection at each step was carried out in the presence of piperidine in DMF two times (20% v/v, 10 times the volume of swelled resin) followed by washing with DMF. Final cleavage of the peptides from the solid support was achieved by using reagent R (TFA/thioanisole/1,2-ethanedithiol/anisole 90:5:3:2 v/v/v/v, 10 times the volume of dry resin) for 2 h. Crude peptides were precipitated by addition of cold diethyl ether (Et₂O), separated, washed by centrifugation (washed with diethyl ether, 3×50 mL and centrifuged at 4000 rpm for 5 min), and were purified by preparative reverse-phase HPLC (Shimadzu LC-8A preparative liquid chromatograph) on a Phenomenex-Gemini C18 column (10 mm, 250×21.2 mm). The peptides were separated by eluting the crude peptide at 12.0 mL/min using a gradient of 5-65% acetonitrile (0.1% TFA) and water (0.1% TFA) over 60 min, and then, they were lyophilized. Chromatograms were recorded at 220 nm using a UV detector. The purity of final products (>95%) was confirmed by HPLC. The chemical structures of compounds were determined by using a SELDI-TOF mass spectrometer on a Ciphergen protein chip instrument using α-cyano-4-hydroxycinnamic as a matrix or a high-resolution Biosystems QStar Elite time-of-flight electrospray mass spectrometer.

### Example 3

### Synthesis of Cyclic Peptide Cys-Lys-Asn-Tyr-Lys-[Lys-Thr-Glu(β Ala)]-Val (NH₂-CKNYK-[KTE(β-A)]V—OH) (4)

Fmoc-Val-Wang resin (1, 3.80 g, 1.25 mmol, 0.33 mmol/g) was swelled under dry nitrogen using anhydrous DMF for about 25 min. The excess of the solvent was filtered off. The swelling and filtration steps were repeated for 2 more times before the coupling reactions. The Fmoc group was deprotected by using 20% piperidine in DMF two times (20% v/v, 2×125 mL, 25 min each) followed by extensive washing with DMF (6×50 mL) Fmoc-Glu(OPhipr)-OH (3 equiv, 1.83 g, 3.75 mmol) was coupled with the amino group of resin by using HBTU (1.42 g, 3.75 mmol, 3 equiv)/DIPEA (6 equiv, 1.31 mL) in DMF (20 mL) for 2 h. A small amount of the resin was subjected to Kaiser test, which showed negative result indicating the coupling was completed. The resin was washed extensively with DMF (6×50 mL). The Fmoc group was deprotected by 20% piperidine in DMF as described above. The subsequent coupling of amino acids, Fmoc-Thr(tBu)-OH, and Dde-Lys(Fmoc)-OH was carried out and finally Fmoc-βAla-OH was coupled to the side chain of K₅ and the resin was washed DMF. The PhiPr group of glutamic acid was removed using cocktail (TFA:ethanedithiol:DCM, 2:5:93, v/v/v, 125 mL) for 4×15 min. The resin was washed with DMF and Fmoc group of the alanine was removed by using piperidine in DMF (20%, 2×25 min, 100 mL). The resin was found to become aggregated due to the presence of positive and negative charged residues and hence was washed with DIPEA in DMF (3×3 min, 25 mL) that resulted in formation of DIPEA salts. After washing the resin with DMF, the solvent was filtered. The resin was allowed to agitate for 30 min in mixture of solvent, DMSO:NMP (1:4, 200 mL). After resin beads looked uniform in the solvent, PyBOP/HOBT/DIPEA (3 equiv/3 equiv/6 equiv, 1.95 g/0.51 g/1.31 mL) were added in the solvent and allowed the resin beads to agitate for 2 h to afford 2. After 2 h, small amount of beads was taken out and washed with DMF, DCM, and ethanol to perform Kaiser test, which showed negative result demonstrating that the cyclization was completed. The formation of cyclic peptide was also confirmed by HR-MS (ESI-TOF): calcd. 692.3745; found 693.4215 [M+H]⁺). After 2 h, the resin was washed with DMF. The Dde group in 2 was removed by using hydrazine in DMF (2%, 3×125 mL). The resin was washed with DMF. Subsequent coupling of amino acid was performed using Fmoc-Lys(Boc)-OH, Fmoc-Tyr(OtBu)-OH, Fmoc-Asn(Trt)-OH, Fmoc-Lys(Boc)-OH, and Boc-Cys(Trt)-OH to yield 3. The resin was washed with DMF (3×125 mL), DCM (3×125 mL), and MeOH (3×125 mL), respectively, and dried in vacuum for 24 h before doing cleavage. The cleavage cocktail, reagent R (TFA/thioanisole/1,2-ethanedithiol/anisole 90:5:3:2 v/v/v/v, 25 mL) for 2 h was added to the resin and was incubated at room temperature for 2 h. The peptide was precipitated with cold ether and lyophilized after dissolving in water. The white dry powder was subjected to purification using semi-prep RP-HPLC by using acetonitrile and water with 0.1% TFA (v/v) with gradient from 0 to 30% of acetonitrile in 40 min to afford building block peptide 4. HR-MS (ESI-TOF): calcd. 1164.5964; found 1165.6649 [M+H]⁺, 583.3380 [M+H/2]²⁺.

### Example 4

### Synthesis of Polyarginine-Cysteine Peptide C(Npys)-(R)₇—CONH₂ (6)

Rink Amide resin (5, 0.5 mmol, 0.40 mmol/g loading, 1.25 g) was swelled in anhydrous DMF under dry nitrogen for 15 min. The excess of the solvent was filtered off. The swelling and filtration steps were repeated 2 more times before the coupling reactions. Fmoc group on the resin was deprotected by using 20% piperidine in DMF (20% v/v, 50 mL, 2×15 min) followed by extensive washing with DMF (7×60 mL). Fmoc-Arg(Pbf)-OH (811 mg, 2.5 eq) was coupled with the amino group of resin by using HBTU (418 mg, 2.2 equiv) and DIPEA (436 μL, 5 equiv) in DMF (30 mL) for 1.5 h. A small amount of the resin was subjected to Kaiser test, which showed the absence of free amino group, suggesting that the completion of coupling. The resin was washed extensively with DMF. The Fmoc group was deprotected by 20% piperidine in DMF (50 mL, 2×15 min) followed by extensive washing with DMF (7×60 mL). Subsequent six more arginine residues were assembled on the resin through coupling of Fmoc-Arg(Pbf)-OH followed by the N-terminal Fmoc group deprotection as described above. The coupling reaction was followed by assembling Fmoc-Cys(Trt)-OH (732 mg, 2.5 equiv) in the presence of HBTU (474 mg, 2.5 equiv, 1.25 mmol) and DIPEA (436 μL, 5 equiv). The N-terminal Fmoc group was deprotected with 20% piperidine in DMF (50 mL, 2×15 min) followed by extensive washing with DMF (7×60 mL). The resin was washed with DMF (3×50 mL), DCM (3×50 mL), and MeOH (3×50 mL), respectively. The resin was dried in vacuum for 24 h, followed by final deprotection and cleavage of the peptide from the resin using the 2,2′-dithiobis(pyridine) (5 equiv, 551 mg) in cleavage cocktail (water/triisopropylsilane/TFA (2.5:2.5:95 v/v/v, 18 mL, 4 h) to afford polyarginine peptide containing activated cysteine after HPLC purification and lyophilization. SELDI-TOF (m/z) [C₅₀H₉₅N₃₁O₈S₂]: calcd. 1321.7421; found 1327.2065 [M+6H]⁺, 1218.3021 [CR₇-Npys]⁺.

### Example 5

### Synthesis of Polyarginine Peptide NH₂—(R)₇—CONH₂

Rink Amide resin (5, 0.5 mmol, 0.36 mmol/g loading, 1.39 g) was swelled in anhydrous DMF under dry nitrogen for 15 min. The excess of the solvent was filtered off. The swelling and filtration steps were repeated 2 more times before the coupling reactions. Fmoc group on the resin was deprotected by using 20% piperidine in DMF (20% v/v, 50 mL, 2×15 min) followed by extensive washing with DMF (7×60 mL). Fmoc-Arg(Pbf)-OH (811 mg, 2.5 equiv) was coupled with the amino group of resin in the presence of 2-(1H-benzotriazole-1-yl)-1,1,3,3-tetramethyluronium hexafluorophosphate (HBTU, 418 mg, 1.1 mmol) and N,N-diisopropylethylamine (DIPEA, 436 μL, 5 equiv) in N,N-dimethylformamide (DMF, 30 mL) for 1.5 h. A small amount of the resin was subjected to Kaiser test, which showed the absence of free amino group, indicating the completion of the coupling. The resin was washed extensively with DMF. The Fmoc group was deprotected by 20% piperidine in DMF (50 mL, 2×15 min) followed by extensive washing with DMF (7×60 mL). Subsequent six more arginine were assembled on the resin through coupling of Fmoc-Arg(Pbf)-OH, followed by the N-terminal Fmoc group deprotection as described above. The resin was washed with DMF (3×50 mL), dichloromethane (DCM, 3×50 mL), and methanol (MeOH, 3×50 mL), respectively. The resin was dried in vacuum for 24 h, followed by final deprotection and cleavage of the peptide from the resin using cleavage cocktail (trifluoracetic acid (TFA)/thioanisole/1,2-ethanedithiol/anisole (90:5:3:2 v/v/v/v, 18 mL, 5 h) to afford polyarginine peptide R₇—CONH₂ after HPLC purification by using a reversed-phase Hitachi HPLC (L-2455) on a Phenomenex Prodigy 10 μm ODS reversed-phase column (2.1 cm×25 cm) and a gradient system, and lyophilization. MALDI-TOF (m/z) [C₄₂H₈₇N₂₉O₇]: calcd. 1109.7343; found 1110.7398 [M+H]⁺.

### General Procedure for Formation of Dicysteine-Polyarginine Peptide Containing Disulfide Linkage.

The final disulfide coupling was performed by using activated polyarginine-cysteine peptide C(Npys)-(R)₇—CONH₂ (20.0 mg, 15.12 mmol) dissolved in 2 mL of degassed water with the addition of equi-molar amount of cysteine or peptides having free thiol group (1.84 mg, 15.12 mmol) at room temperature. After addition of thiol containing compound, the color of reaction was turned to light yellow. After stirring for 18-36 h, the progress of reaction was monitored using MALDI-TOF to monitor the completion of the reaction. The reaction mixture was diluted with ethyl acetate (2 mL), and the aqueous phase was separated and extracted with ethyl acetate (3×5 mL). The aqueous phase was lyophilized, and the residue was purified by reverse phase HPLC using 1-20% acetonitrile gradient over 30 min to afford dicysteine-polyarginine peptide containing disulfide linkage in about 35-60% overall yield. The chemical structure of the final product was determined by using a high-resolution time-of-flight electrospray mass spectrometer.

### Synthesis of CN-2097 (7) by Solution-Phase Coupling Reaction of 4 and 6

The final disulfide coupling was performed by using polyarginine peptide (6, 20.0 mg, 15.12 mmol) dissolved in 2 mL of degassed water and addition of peptide 4 (17.6 mg, 15.12) at room temperature. After addition of cyclic peptide 4, the color of reaction was turned to light yellow. After stirring for 18 h, the reaction was diluted with ethyl acetate (2 mL). The aqueous phase was separated and extracted with ethyl acetate (3×5 mL). The aqueous phase was lyophilized and the residue was purified by reverse phase HPLC (C18 column using 1-20% acetonitrile gradient over 30 min) to afford CN2097 (7) in 60% overall yield (FIG. 1). The chemical structure of CN2097 was determined using a high-resolution time-of-flight electrospray mass spectrometer. SELDI-TOF: calcd. 2375.32; found 2380.40 [M+5H], 1217.60 [M+H−cyclic]⁺, 1170.60 [M+H−CR7]⁺.

### Example 5

### Optimization of Macrocyclic PDZ Domain Inhibitors

The high affinity macrocycles known for targeting PDZ domain were synthesized from the peptide, KNYKKTEV, based on the C-terminal residues of CRIPT by cyclization between side chain of the glutamic acid (2) and lysine side chain (4) residues via β-alanine linkage as mentioned in background section. Unfortunately, attempts to improve the biological profile of these macrocycles have so far met with little success. To improve the therapeutic profile, these macrocycles require to be the cell permeable and stable in the presence of blood plasma. These macrocycles are water soluble and limited cellular uptake. Thus, various drug delivery approaches will be used to enhance cellular uptake as well as the stability of the lead macrocyclic molecule. The structural modifications will also establish the structure-activity relationship for designing of small molecule for future prospect of this proposal.

The optimization of macrocyclic PDZ domain inhibitor will be carried out by the various methods described below. The structure-activity relationships will be established and the hit compounds will be selected for further conjugation.

The synthesis of cyclic peptide using “D”-amino acid for several derivatives of peptides containing D-amino acid have been synthesized. Using D-amino acid in the optimization, the lead compound has stability towards the proteolytic degradation. Macrocycles are synthesized by using step by step changes in one or more L-amino acid to D-amino acid inside the macrocycle ligand and then evaluating the effect of these unnatural amino acids on the affinity for PDZ domain.

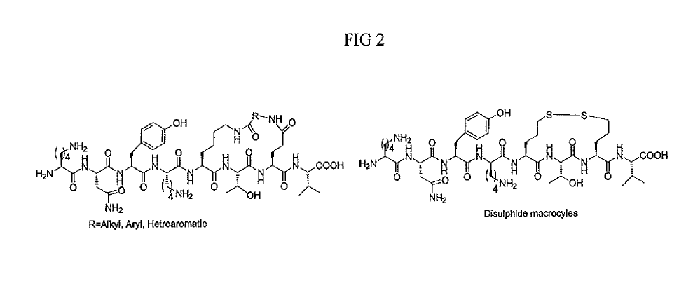

Example of the modification of conformationally constrained molecules used the cyclic peptide, K₁N₂Y₃K₄K₅[β-A-T₆E₇]V₈, based on the C-terminal residues of CRIPT, was synthesized based on the fact that the only cyclic peptide with side chain from lysine and glutamic acid cyclized generates potent and biological active cyclic peptide PDZ domain inhibitory activity. The other cyclic peptide generated either from side chain to N-terminal or Side chain to C-terminal showed no inhibitory effect or they were not potent against PDZ domain. Various different approaches of cyclization will be used like (N-terminal to side chain, side chain to side chain, C-terminal to side chain) and different sizes of the cyclic ring to provide various new conformation in the sequence of the lead peptide (FIG. 2). The other cyclic peptide generated either from side chain to N-terminal or Side chain to C-terminal showed no inhibitory effect or they were not potent against PDZ domain. The different sizes (number of carbon atom) of the cyclic ring is reported, which showed no need for further optimization of cyclic peptide by either of these method.

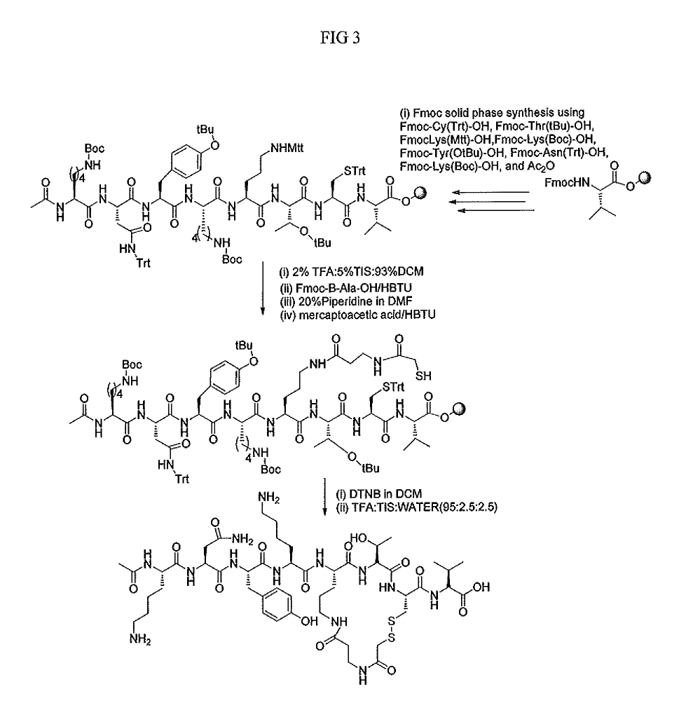

Another example of the optimization in this invention is to use disulfide cyclization to make a conformationally constrained peptide containing all other amino acids in sequence without any modification. The disulfide cyclization step is challenging and tedious and needs more time. FIG. 3 is a detailed synthetic methodology for the synthesis of a disulfide analogue of macrocyclic peptide.

The unique elements of the disulfide bond in this cyclic peptide provided an opportunity to couple an analog with PDZ domain inhibitory activity with a significant effect on various cell signaling pathway. The CN2097 peptide is a polyarginine disulfide with the cyclic peptide, of which it was found that disulfide bond is important for the activity of the CN2097. The new disulfide bond macrocycle produced a new conformation and the disulfide linkage will give new pharmacophore and have a different activity profile against the PDZ domain. The design synthetic is easy to scale up out to provide good abundant amounts of the peptide for assays. From this approach, a variety of new derivatives is generated with the variation of carbon chain length in the cyclic part of disulfide linkage

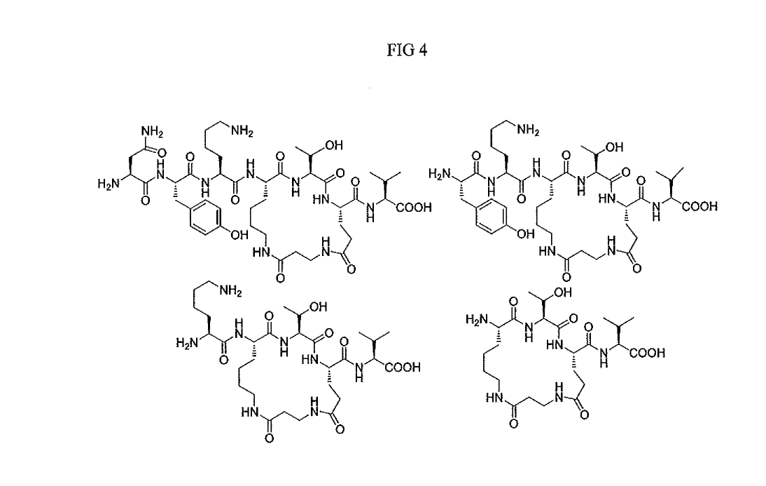

The peptidic nature of macrocyclic ring is reduced by removing one amino acid at a time from N-terminal to the sequence of the cyclic peptide. The affinity of these peptides against PDZ domain needs to be investigated. The structure of examples of synthesized peptides is given in FIG. 4.

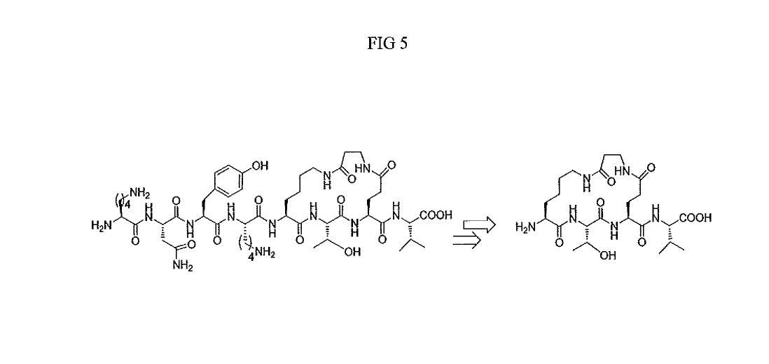

By truncation from N-terminal to C-terminal, the peptidic nature of macrocycle is reduced as shown in FIG. 5. Using alanine scanning throughout the macrocyclic is to provide the essential amino acid for binding and minimize the peptidic nature of the structure.

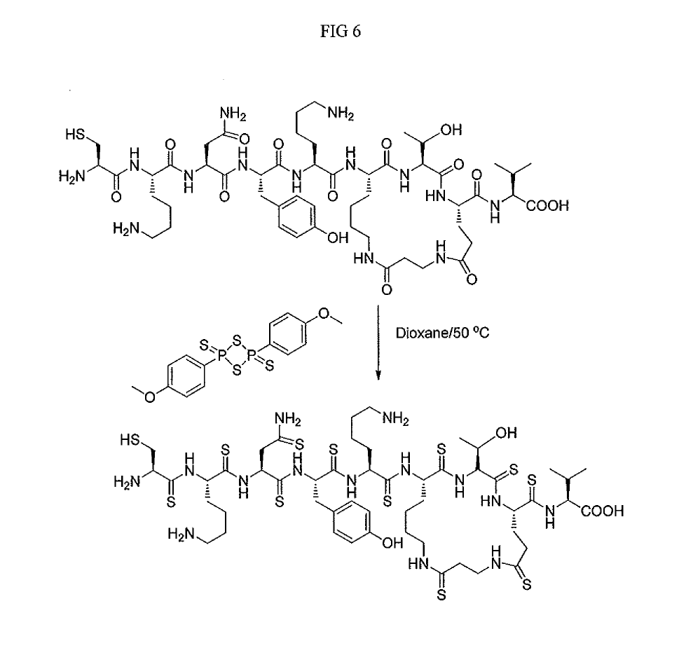

The modification of peptidic bond to ketone (COCH₃), thioamide (CS—NH) or reverse amide (NH—CO), is to produce compounds with less peptidic nature and thus improved stability. An example of the modification of the peptidic bond to thioamide is shown in FIG. 6. Large amount of cyclic peptide was prepared which is used to modify the amide bond of the peptide as follows. 100 mg of HPLC purified cyclic peptide was made to react with the Lawesson's reagent [2,4-bis(4-methoxyphenyl)-1,3-di dithio-2,4-diphosphetane-2,4-dithione] in dioxane to make the thioamide derivative of the cyclic peptide. Alternatively, a large amount of cyclic peptide is required for sulfurization of the peptide with Lawesson's reagent due to problem of regioseletivity and yield.

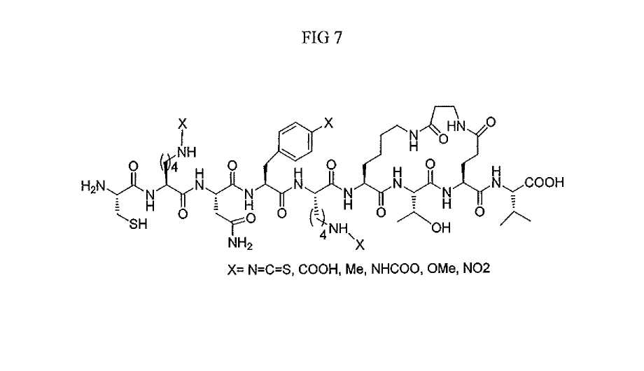

Various functional group substitution (—N═C═S, —COOH, -Me, —NHCOO, —OMe, —NO₂) modifying the macrocyclic results in changes in electronegativity, size of functional groups, and other physicochemical properties impacting its inhibitory activity (FIG. 7).

### Example 6

### Conjugation of Macrocyclic PDZ Domain Inhibitors with Other Compounds

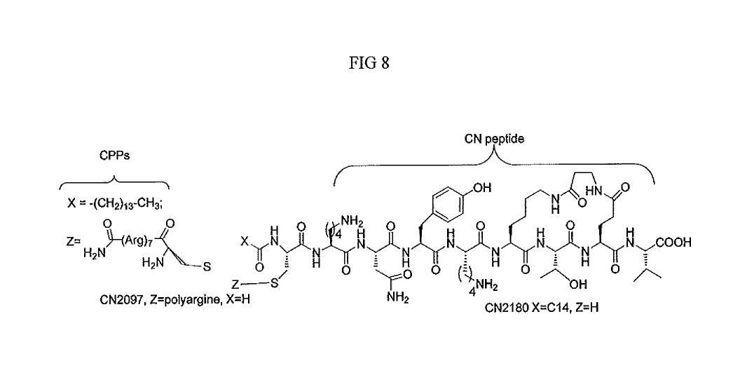

An example of lipidation of macrocyclic PDZ domain inhibitors is performed as follows. The myristoyl derivative CN2180 was prepared by N-terminal acylation using myristic anhydride (FIG. 8). Other fatty chains (C16-22) derivatives of the lead macrocyclic compounds are synthesized to alter lipophilicity with cellular permeability and biological activity.

Yet another modification is conjugation of macrocyclic PDZ domain inhibitors with cell-penetrating peptides (CPPB) (CN2097). In this example, polyarginine-based peptides (7-9 residues) are used to synthesize cell permeable conjugates of lead PDZ domain inhibitors. The linking of 7th residue of arginine peptide with a hydrolyzable linker (disulfide bond) to the macrocycle, (CN2097, FIG. 1) has been reported inside neuronal cells. Further, unnatural amino acid derivatives of polyarginine is expected to have similar applications with resistance towards proteolytic degradation.

Still another modification is a Szeto-Schillar peptide conjugate of macrocyclic peptide (Szeto-CN2097). Because there is of great interest to deliver the peptide intracellularly, the szeto-schillar peptide (H-Dimethyl tyrosine-M-Arg-Phe-Lys-NH₂) was found to target mitochondria inside the cells. This sequence has two unnatural amino acid (e.g., dimethyl tyrosine and D-arginine), which make the peptide more resistant to protease degradation.

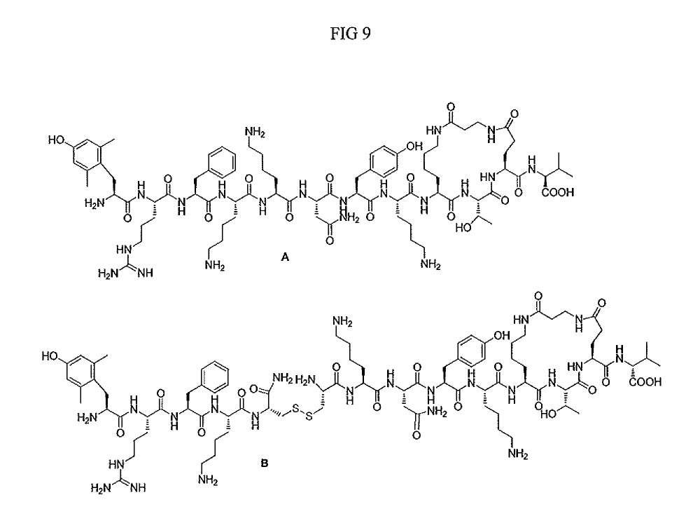

The szeto-Schillar peptide was synthesized by Fmoc-t/Bu solid phase synthesis (FIG. 9). The szeto peptide sequence was conjugated using amide bond at the N-terminal of the cyclic CN peptide (Szeto-CN) or using a cysteine disulfide bond with a cysteine at the C-terminal of szeto sequence (Szeto-CN2097). These derivatives provide smaller size, low molecular weight, and different target as compared to CPPs.

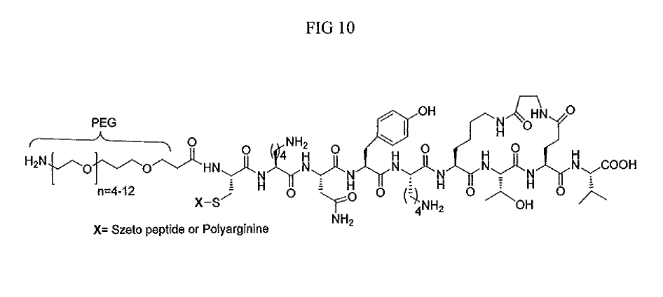

Yet another modification is the pegylation to improve macrocycle stability in the blood. The corresponding derivative of CN2097 or szeto-CN2097 (FIG. 10) is synthesized with N-terminal pegylation to enhance stability in the plasma. The pegylation acts to form a protective “shell” around the peptide. This shell and it's associated waters of hydration shield the peptide from immunogenic recognition and increase resistance to degradation by proteolytic enzymes such as trypsin, chymotrypsin and streptomyces griseus protease.

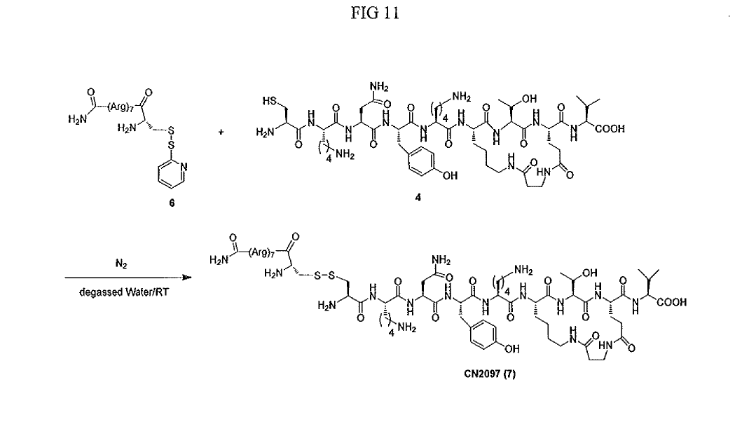

Standard Fmoc-based protocols is used to synthesize macrocycles that target the PDZ domain of PSD-95 as previously reported. The peptide, KNYKKTEV, based on the C-terminal residues of CRIPT, was cyclized between the glutamic acid and lysine side chain residues via β-alanine linkage. The peptide was either myristoylated (CN2180) or linked to a polyarginine tail (CN2097; FIG. 1) using the cysteine side chain to enhance its uptake by neurons. Also shown is a control cyclic peptide, CN3200, having the Ala/Ala double mutation at the 0/−2 positions, which knocks out binding to PDZ1-2 and PDZ3 domains. FIG. 11 shows the synthesis of lead macrocyclic CN2097.

Potential Difficulties and Alternative Approaches.

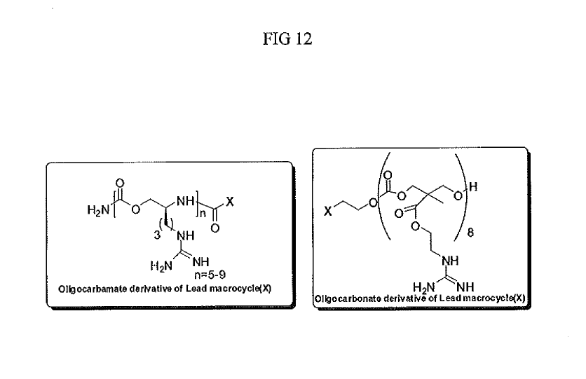

Several derivatives of these macrocycles have been identified for their potential use to target the PDZ domain e.g. CN2097, szeto-CN2097, and biotinylated-CN2097. The lead macrocycle needs to be optimized or converted to small drug like molecules. The alternative method for transporting these macrocycles will be to use multiple guanidinium compounds. The oligocarbamate and oligocarbonate have been found to be as efficient molecular transporters as compared to oligoarginine. They will be non-amide carrier for the transport of cyclic CN compounds intracellularly and even better than (Arg), oligomers. These oligocarbamates will be best alternative carriers as compared to oligoarginine due to their resistance to proteolytic degradation (FIG. 12).

The stability of CN2097 and Szeto CN2097 derivatives in blood was determined as follows.

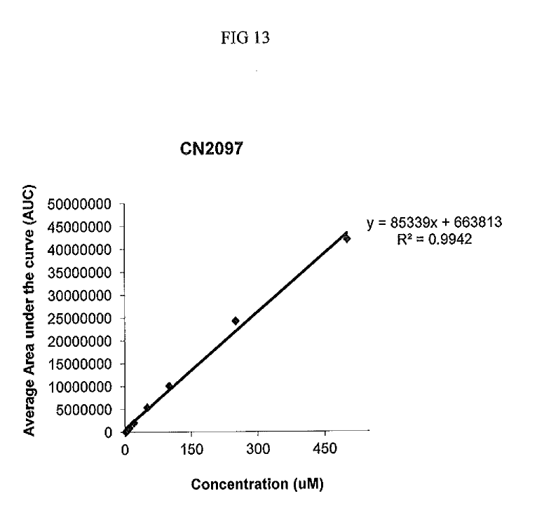

The detection limit of CN2097 was measured by HPLC analysis. Various concentrations of CN2097 were prepared in PBS and injected in HPLC and monitored at 214 nm. The HPLC analysis of CN2097 using 50 microliter injection and detecting up to 500 micromolar. The lowest level of detection was at 2 micromolar, corresponding to 0.1 nmole or 237.68 ng (FIG. 13).

Plasma stability, or T_{1/2}, of CN2097 and szeto-cyclic peptide were compared to the cyclic peptide as shown in Table 1. The T_{1/2} is the time by which the compound was degraded by enzyme to the 50% of original amount. It was demonstrated that the modification of CN2097 and a derivatives increased its stability by about two fold.

TABLE 1
T_{1/2} values for CN2097 and derivatives in the blood plasma.

| S. No. | Peptide | T_{1/2} |
|---|---|---|
| 1. | CN (cyclic peptide with Cysteine) | ~28 min |
| 2. | CN2097 (polyarginine-disulfide-cyclic peptide) | ~48.2 min |
| 3. | Sezto-CN (sezto- disulfide cyclic peptide) | ~43.2 min |

### Example 7

### Synthesis of Novel Polyarginine Disulfide Peptides to CN2097

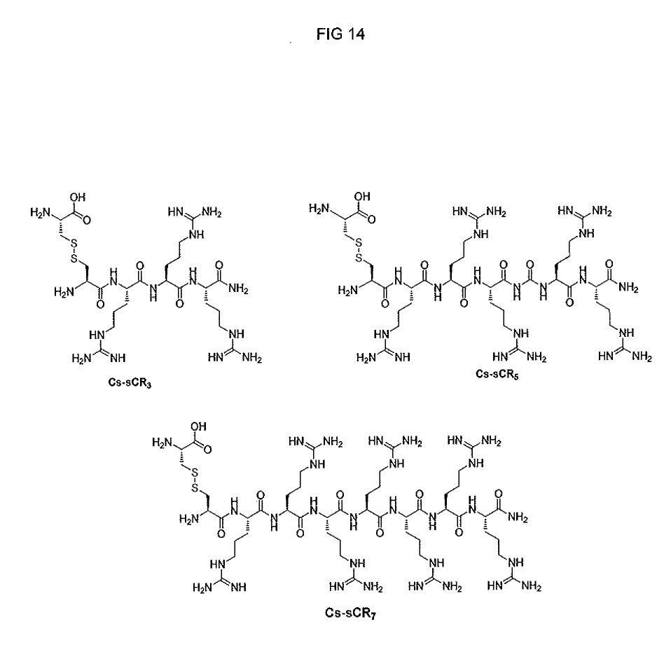

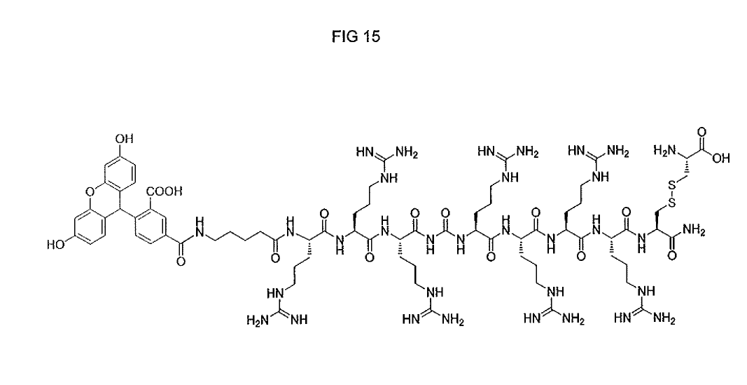

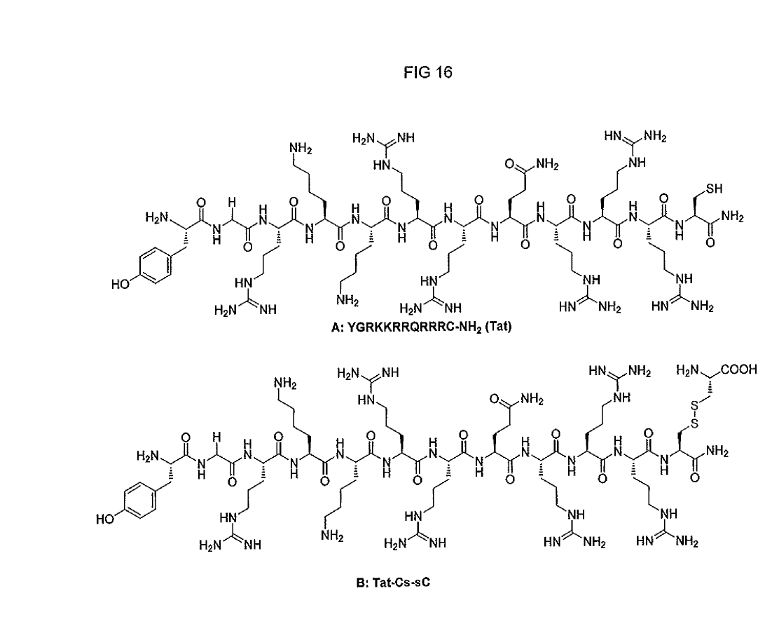

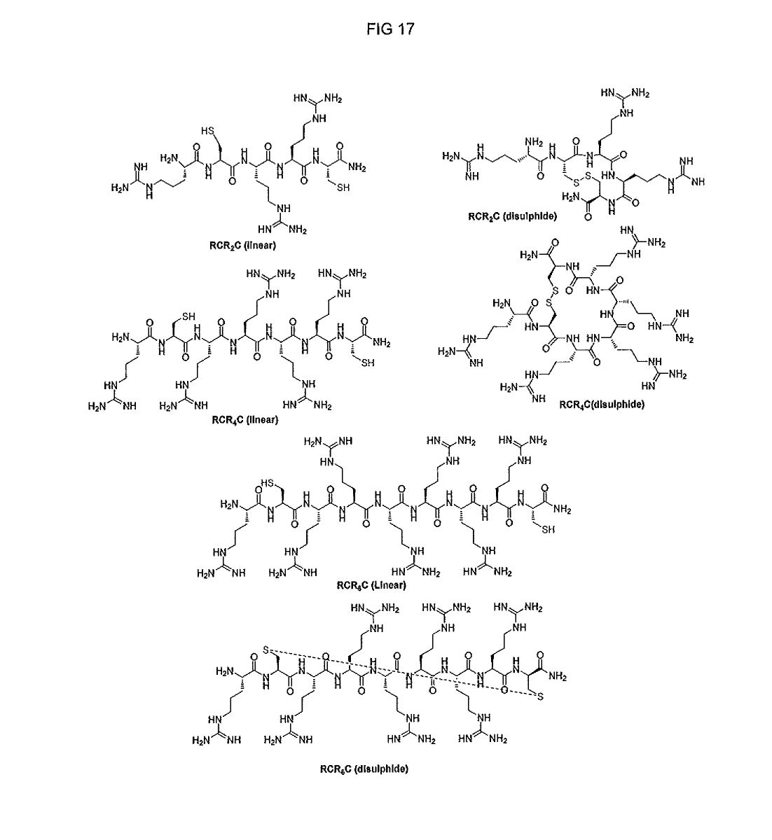

To expand the potential of CN2097 against retinal ganglion cells, new polyarginine disulfide peptides were synthesized (FIG. 14-17).

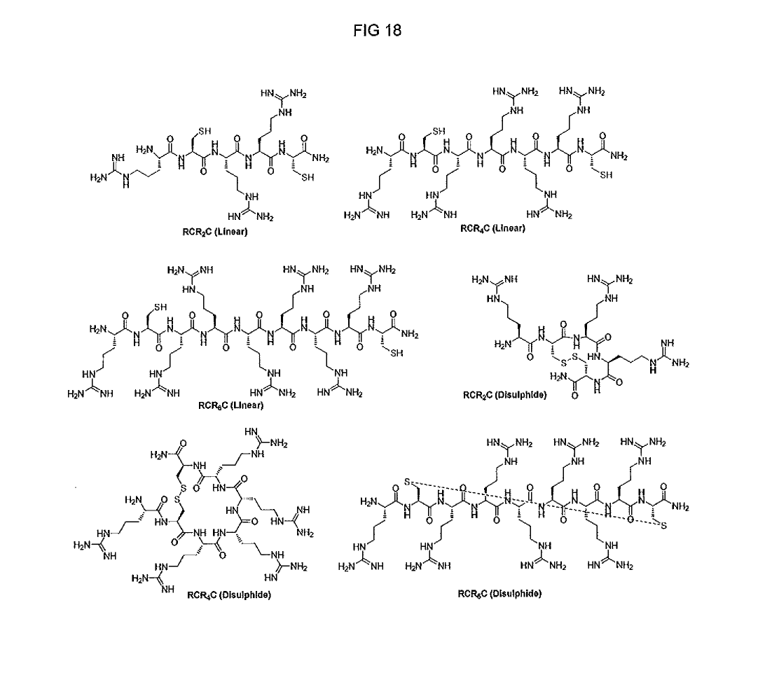

In addition, to optimize the position of cysteine and arginine residues for generating neuroprotective peptides, the number and position of cysteine and arginine residues and sequence of the peptide is modified. Arginine and cysteine residues can be “walked down” each position of the peptide to determine optimal sites for generating full protection. Peptide analogues are modified all possible combinations of R and C are incorporated into the parent structure that include, R₉C, R₉—Cs-sC, R₈—C—C, R₈—Cs-sC, R₇—C—C, R₇—Cs-sC, R₇C, R₆CR, R₆Cs-sCR, R₆C—C—R, R₂CR₅, R₅—Cs-sC-R₂, R₅—C—C—R₂, R₄CR₃, R₄Cs-sC-R₃, R₄C—C—R₃, R₃CR₄, R₃Cs-sC-R₄, R₃C—C—R₄, R₂CR₅, R₂Cs-sC-R₅, R₂C—C—R₅, R₁CR₆, R₁Cs-sC-R₆, R₁C—C—R₆). The optimal position of cysteine residues (N-terminal, C-terminal or between two arginine residues), number of required arginine or cysteine residues, and requirement of disulfide bond or amide bonds between cysteine residues are synthesized. Further, the modification in the peptide sequence is carried out to see the effect of the disulfide bond as well as number of arginine residue in the sequence. Alternatively, a new series of peptide sequence RCRₙC where n=2-6 may be provided. The peptide is synthesized with or without disulfide bond (FIG. 18).

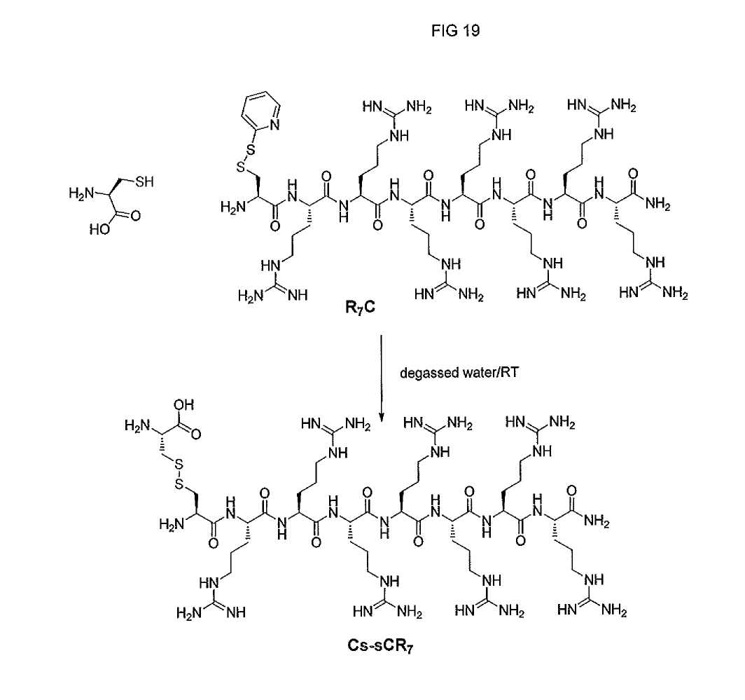

Standard Fmoc-based protocols are used to synthesize the peptide (FIG. 19). The synthesized peptides are further optimized with systemic structure modifications to develop the compounds with lower molecular weights and less peptidic characters. The peptide is optimized by truncation from N-terminal to C-terminal, by use of unnatural amino acid (D amino acid), and by changing the peptide backbone to ketone (COCH₃), thioamide (CS—NH) or reverse amide (NH—CO). These proposed structural modifications provide new insights into development of the potent neuroprotective compounds. After initial lead compounds are discovered, they are conjugated with cell-penetrating functional groups for enhancing their cellular uptake.

Pegylation is carried out by using various PEG molecules of different lengths (4-8) to afford stable derivatives of the lead non peptidic compounds. Pegylation helps not only the delivery of the therapeutic molecule, also changes the pharmacokinetics, and pharmacodynamics of the molecules. Pegylation may decrease cellular peptide clearance by reducing elimination through the reticuloendothelial system by specific cell-protein interaction.

Lipidation at amino group has been applied to lead compounds using myristoyl (C14 carbon chain) as fatty chain to enhance cellular permeability. Conjugation with various lipophilic fatty acyl chains (C12-C20) (lipidation) offers an attractive method to improve the cell permeability. The lipophilic chain is hydrolyzed intracellularly by hydrolytic enzymes and the active parent analog is released.

The szeto-schillar peptide (H-dimethyl tyrosine-[D]-Arg-Phe-Lys-NH₂) has been found to target mitochondria inside the cells. Linking of the peptide to PDZ inhibitor macrocycle using a similar disulfide linkage is investigated. The szeto-CN2097 conjugate is a lower molecular weight and different biological profile versus conventional cell-penetrating peptide derivative e.g. CN2097.

The optimization of peptide is carried out by the various methods described below. The structure-activity relationships is established and the hit compounds are selected for further conjugation.

By using D-amino acid in the optimization, the lead compound is stable towards the proteolytic degradation. Peptidomimetic is synthesized by using step by step changes in one or more L-amino acid to D-amino acid inside the peptide ligand and then is evaluated the effect of these unnatural amino acids for their neuroprotection.

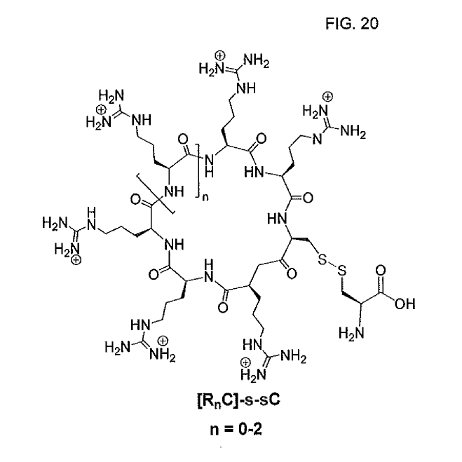

Various different approaches of cyclization is used in the peptide like (N-terminal to side chain or lysine instead of arginine, side chain to side chain, C-terminal to side chain) and different sizes of the cyclic ring (with linker) to provide various new conformations in the sequence of the lead peptide. Some of the proposed structures are: [RₙC]-s-sC (FIG. 20), [Rₙ—C—C], [Rₙ—C]C, [Rₙ—C-s-s-C], s-sC, and Rₙ[K—C]-s-sC.

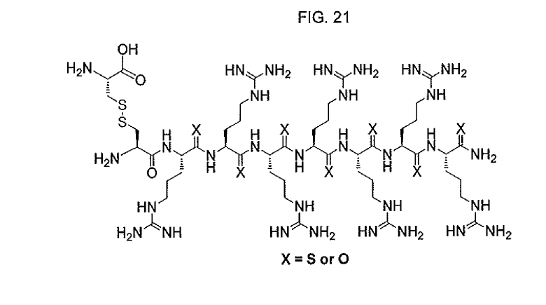

By truncation from N-terminal to C-terminal, the peptidic nature is reduced. Using alanine scanning throughout the peptide provides the essential required amino acid for their neuroprotection and minimize the peptidic nature of the structure. The modification of peptidic bond to ketone (COCH₃), thioamide (CS—NH) (FIG. 21) or reverse amide (NH—CO) generates compounds with less peptidic nature and thus improves their stability.

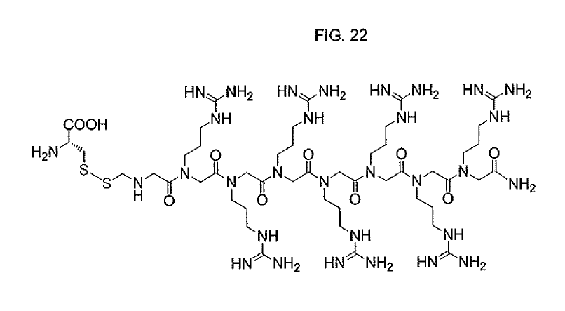

Peptoids are another examples of peptidomimetics in which the side chains of amino acids are appended to the nitrogen atom of the peptide backbone and α-carbon atoms are free which resulted in the complete resistance towards proteolysis and also are not subjected to denaturation with the solvent, temperature and urea (FIG. 22).

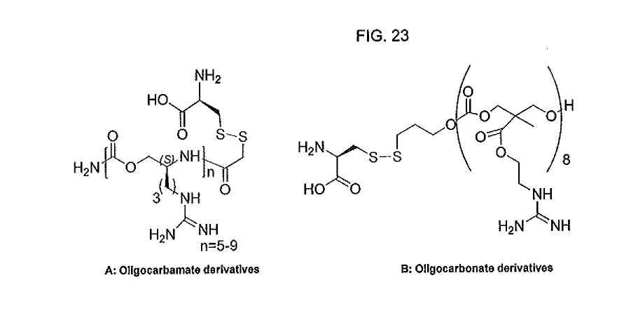

The oligocarbamate and oligocarbonate have been found to be as efficient molecular transporters as compared to oligoarginine, can be used to assay for their neuroprotective properties. They are able to pass the cell membrane even better than (Arg), oligomers. These oligocarbamates are alternative molecules to polyarginine due to their resistance to proteolytic degradation (FIG. 23).

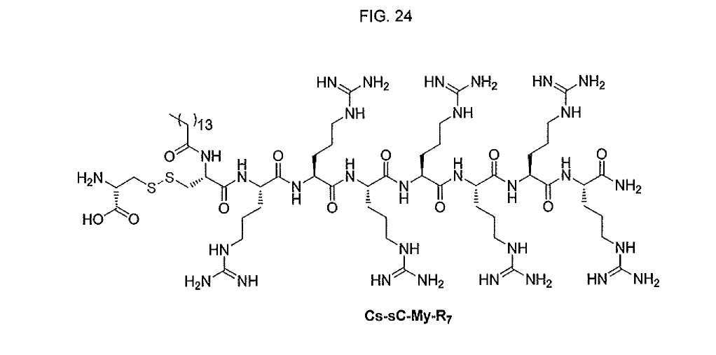

The myristoyl derivative of lead peptide is prepared by N-terminal acylation using myristic anhydride (FIG. 24). Other fatty chains (C16-22) derivatives of the lead peptide compounds is synthesized to correlate the lipophilicity with cellular permeability and biological activity.

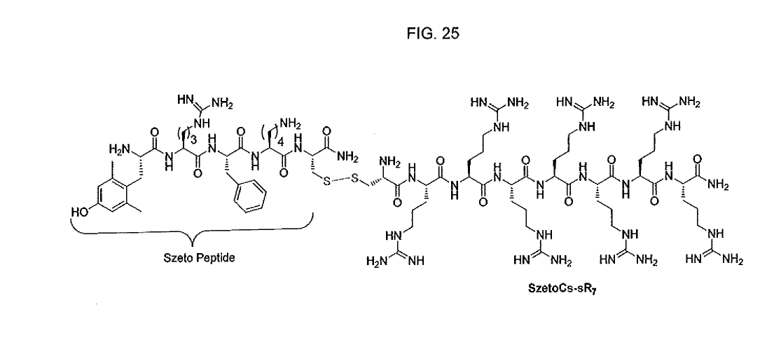

The szeto peptide sequence is conjugated with using amide bond at the N-terminal of the peptide through using a cysteine disulfide bond with a cysteine at the C-terminal of szeto sequence (Szeto-R₇C) (FIG. 25). These derivatives provide smaller size, low molecular weight and different target as compared to CPPs.

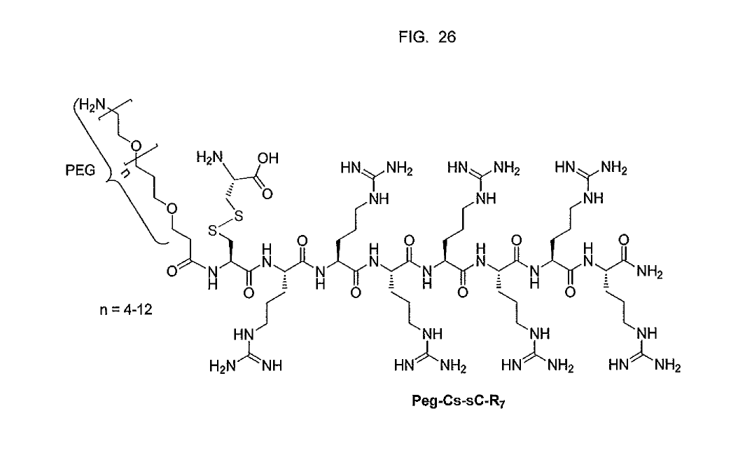

The corresponding peglated derivative of R₇Cs-sC (FIG. 26) is synthesized with N-terminal pegylation to afford enhanced stability in the plasma. The pegylation forms a protective “shell” around the peptide. This shell and it's associated waters of hydration shield the peptide from immunogenic recognition and increase resistance to degradation by proteolytic enzymes such as trypsin, chymotrypsin and streptomyces griseus protease.

Those skilled in the art will appreciate that numerous modifications and variations may be made to the above disclosed embodiments without departing from the spirit and scope of the inventions.

1. A compound comprising at least one substituted macrocyle selected from the group consisting of Formulas I-III:

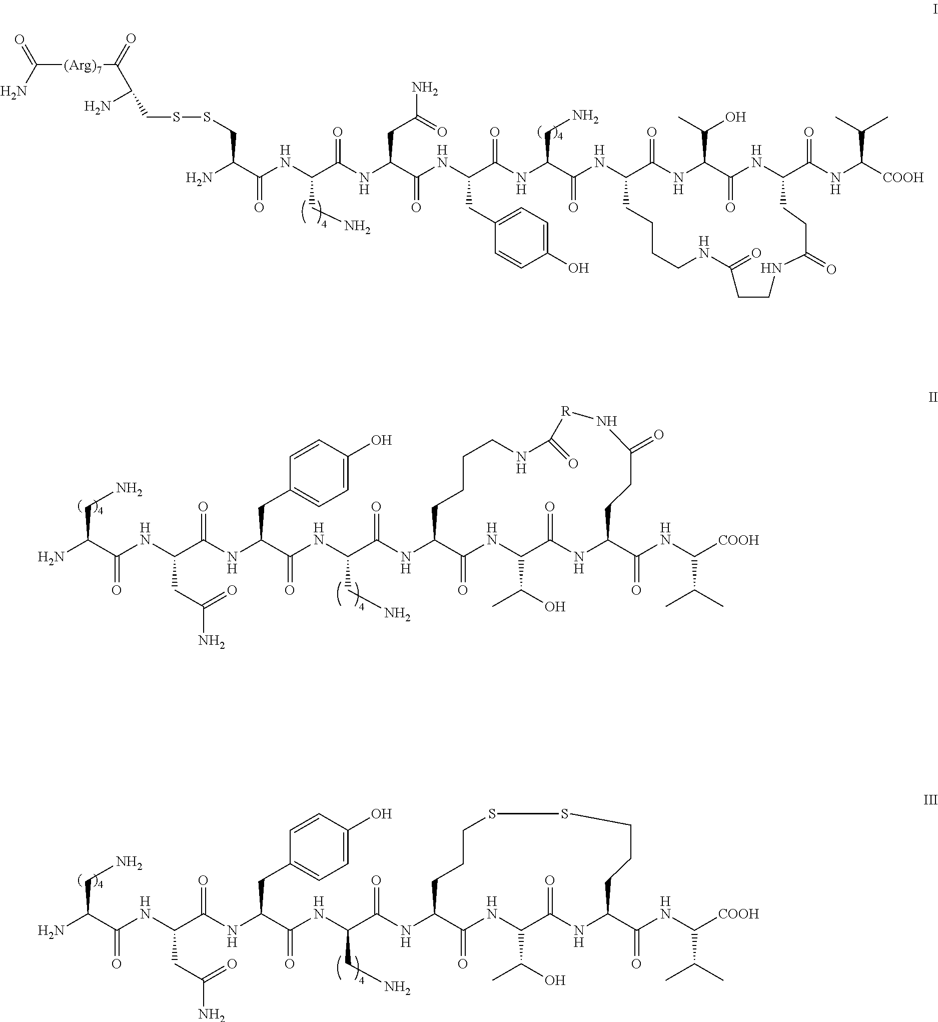

2. The macrocyle compound of claim 1, wherein Formulas 1 is reduced by one amino acid selected from the group consisting of Formulas IV-VI:

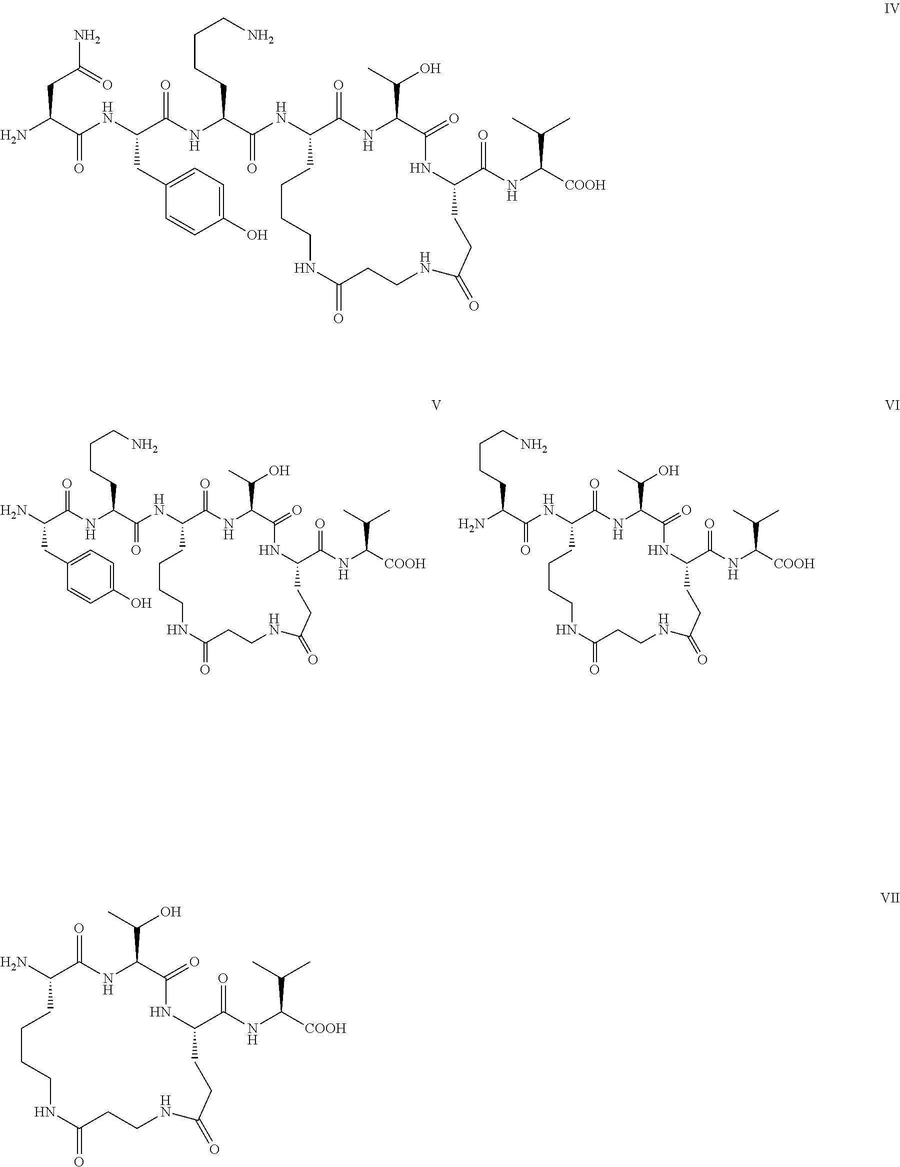 [SMILES: CC(C)[C@H](NC(=O)[C@@H]1CCC(=O)NCCC(=O)NCCCC[C@H](NC(=O)[C@H](CCCCN)NC(=O)[C@H](Cc2ccc(O)cc2)NC(=O)[C@@H](N)CC(N)=O)C(=O)N[C@@H](C(C)O)C(=O)N1)C(=O)O ; CC(C)[C@H](NC(=O)[C@@H]1CCC(=O)NCCC(=O)NCCCC[C@H](NC(=O)[C@H](CCCCN)NC(=O)[C@@H](N)Cc2ccc(O)cc2)C(=O)N[C@@H](C(C)O)C(=O)N1)C(=O)O ; CC(C)[C@H](NC(=O)[C@@H]1CCC(=O)NCCC(=O)NCCCC[C@H](NC(=O)[C@@H](N)CCCCN)C(=O)N[C@@H](C(C)O)C(=O)N1)C(=O)O ; CC(C)[C@H](NC(=O)[C@@H]1CCC(=O)NCCC(=O)NCCCC[C@H](N)C(=O)N[C@@H](C(C)O)C(=O)N1)C(=O)O]

3. A compound comprising a substituted macrocyle of Formula VIII, wherein

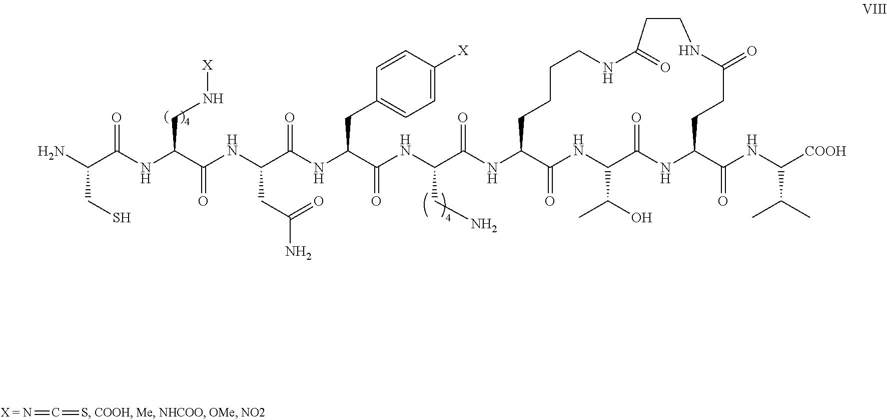

4. The macrocyle compound of claim 1, wherein Formulas 1 is modified selected from the group consisting of myristoylated, polyarginine-based peptides, Szeto0Schillar peptide, N-terminal pegylation, KNYKKTEV, oligocarbamate, oligocarbonate, and oligoarginine.
